(19) United States
(12) Patent Application Publication
(10) Pub. No.: US 2012/0143087 A1
(43) Pub. Date: Jun. 7, 2012

(54) FETAL LACTIC ACID MONITOR

(76) Inventors: Zeev Ganor, Herzliya (IL); Alexander Schoenfeld, Herzlia Pituach (IL); Herzel Laor, Boulder, CO (US)

(21) Appl. No.: 13/376,845

(22) PCT Filed: Jun. 9, 2010
(86) PCT No.: PCT/IL2010/000456

§ 371 Date: Feb. 26, 2012

Related U.S. Application Data
(60) Provisional application No. 61/185,234, filed on Jun. 9, 2009.

(51) Int. Cl. A61B 5/155

(52) U.S. Cl. 600/584

(57) ABSTRACT

A device and method for automatically and repeatedly measuring fetal Lactic Acid (LA) concentration in the blood, for monitoring fetal stress. The device is connected by a probe to the scalp of the fetus and measures the Lactic Acid concentration in the blood at preset intervals. The device is optionally provided with an alarm which is activated when lactic acid concentration is outside a preset normal range.

### FIELD OF THE INVENTION

The present invention relates generally to monitoring devices, and more particularly, to a device for assessing fetal stress by automated, repeated measurement of the amount of lactic acid in fetal blood.

### BACKGROUND OF THE INVENTION

During labor prior to birth, the fetus may experience stress, which requires close monitoring. Currently, fetal stress is generally monitored using heart rate monitors or by measuring the blood oxygen level of the fetus. Unfortunately, neither of these methods accurately indicates whether the fetus is truly in stress and if intervention is necessary, e.g. by caesarian section. These methods may result in false positives which lead to unnecessary surgery, thereby placing the mother and fetus at risk, or false negatives which can result in brain damage and even death of the fetus. Furthermore, a clinical test aimed at establishing the relationship between blood oxygen level and fetal stress failed to show any correlation between the two, as described by Nordstrom L and Arulkumaran S, Obstet Gynecol Sury 53(10): 645-57, 1998; and Kruger K, et al, Am J Obstet Gynecol 181(5 Pt 1): 1072-8, 1999.

Lactic acid (LA) is mainly produced in muscle cells and red blood cells. It is formed by glycolysis, when the body breaks down carbohydrates to use for energy during times of low oxygen levels.

It is known that lactic acid concentrations of at least 4.2 mMol/L in fetal blood are a reliable indicator of fetal stress (Allen et al, Aust NZ J Obstet Gynecol 44(6): 549-552, 2004), and lactic acid concentrations of at least 4.8 mMol/L are an indicator of asphyxia (Kruger K, et al, Am J Obstet Gynecol 181(5 Pt 1): 1072-8, 1999).

If the attending physician is aware of the fetal LA level and its fluctuations throughout the birth, he can decide on proceeding with a cesarean section when the fetus is in stress. However, known methods of measuring fetal blood lactic acid concentrations involve discrete test procedures, which must be individually repeated, requiring an incision each time a sample is withdrawn if repeated measurements are required, which is traumatic and painful for the fetus.

Periodic blood sample units are known in the art, for example the AccuSampler® manufactured by DiLab, Sweden, and are also disclosed in Hansson, et al. U.S. Pat. No. 7,258,672, Gilcher, et al. U.S. Pat. No. 6,113,554 and Blake, et al. U.S. Pat. No. 6,736,783.

Therefore, it would be desirable to provide a device to automatically and repeatedly monitor fetal lactic acid as an indicator of fetal stress. Such a device may help to ensure the use of appropriate medical procedures during labor.

There is thus a need for an accurate and reliable method for monitoring fetal stress during labor.

### SUMMARY OF THE INVENTION

Accordingly, it is a principal object of the present invention to overcome the limitations of prior art fetal stress monitoring techniques by providing, in accordance with the present invention, a device for automated, repeated monitoring of fetal blood lactic acid concentration as an indicator of fetal stress.

The device and method of the present invention provide an accurate and reliable indication of fetal stress, based on measurements which may be automatically repeated throughout labor, requiring minimal amounts of blood to be drawn from the fetus, and without requiring repeated incisions in order to obtain blood samples.

According to some embodiments, there is provided a device for monitoring fetal stress by automatically and repeatedly measuring a lactic acid concentration in a blood sample from a fetus, the device comprising a blood sampling assembly for attachment to a fetal organ to extract a microvolume blood sample; and a test assembly for quantifying the lactic acid concentration in the blood sample extracted by the blood sampling assembly. The test assembly comprises a reaction location in fluid communication with the blood sampling assembly and a reagent reservoir in communication with the reaction location. The reagent reservoir comprises a reagent sensitive to lactic acid concentration, wherein interaction between the blood sample and the reagent provides a measureable indication of blood lactic acid concentration. The device is configured for automatically refreshing the reaction location with a blood sample through the blood sampling assembly and for automatically refreshing the reaction location with reagent from the reagent reservoir.

In some embodiments, the blood sampling assembly and the test assembly are provided as separate units. In some such embodiments, the device further comprises a blood input tube for providing fluid communication between the blood sampling assembly and the test assembly.

In some embodiments of the device of the present invention, the blood sampling assembly is contained within a probe for contacting the blood sampling assembly with the fetus.

In some embodiments, the blood sampling assembly and test assembly are provided within the probe.

In some embodiments, the reaction location is provided proximal to the blood extraction assembly.

In some embodiments, the reagent reservoir comprises a reservoir for containing a liquid reagent. Optionally, in such embodiments, the reaction location comprises a reaction chamber provided within the test assembly.

Alternatively, in some embodiments, the reagent reservoir comprises a reel of test strip wherein the reagent is incorporated into the test strip. In such embodiments, the reaction location is a portion of test strip which contacts a blood sample from the blood extracting assembly. The reel of test strip is optionally disposed on at least one spool for providing continuous feed of unused test strip. Further optionally in such embodiments, the said test assembly further comprises a second spool wherein portions of the test strip are wound onto said second spool after use.

In some embodiments, a distal end of the blood extracting assembly comprises at least one sharp hollow pin for reversible attachment to the body of a fetus. Preferably, the pin has an internal diameter in the range of from about 100 to about 200 micrometers. Optionally, the pin is coated with an anti-coagulant to prevent blood clotting.

In some embodiments, the device is preset to withdraw a blood sample of specified volume, such as, for example, in the range of from about 5 μl to about 7 μl from the fetus via the hollow pin.

In some embodiments, the device is preset to repeatedly withdraw a blood sample from the fetus at specified time intervals, such as, for example in the range of from about 0.5 minutes to about 20 minutes. Preferably the time interval is about 5 minutes.

In some embodiments, the blood sampling assembly comprises at least one blood pump for pumping blood from the at least one pin to the blood sampling assembly.

In some embodiments, the test assembly further comprises a reagent pump for pumping liquid reagent from the reagent reservoir to the reaction location.

In some embodiments, the device further comprises a cleaning fluid reservoir in fluid communication with the blood input tube, wherein the cleaning fluid from the cleaning fluid reservoir flushes blood residues from the blood input tube after passage of the blood sample. Preferably, the cleaning fluid is saline.

In some embodiments, the device further comprises a saline reservoir, a saline input tube in fluid communication with the blood input tube, and a saline pump for pumping saline from the saline reservoir to the blood input tube. Preferably, at least one of the blood pump, reagent pump and saline pump have diameters in the range of from about 3 to about 5 mm. Preferably, at least one of the blood input tube and saline input tube has a diameter in the range of from about 1 mm to about 1.5 mm.

In some embodiments wherein the device comprises a saline reservoir and saline input tube, the blood sample entering the blood input tube is pushed along the blood input tube to the test assembly by saline pumped from the saline reservoir into the blood input tube.

In some embodiments, the blood input tube comprises an open end adjacent to the test strip, and the test unit further comprises a suction pump for removing liquid from the blood input tube via a clearing tube having an opening at a branch point with the blood input tube. The test assembly further comprises an optical detector which distinguishes between saline and blood, such that when the optical detector identifies the presence of the blood sample, a signal is transmitted to the suction pump to cease operation once the blood sample is calculated to have reached the opening at the branch point with the blood input tube, such that the blood sample is allowed to flow to the open end of the blood input tube and contact the test strip at the reaction location.

In some embodiments wherein the reagent reservoir comprises a reel of test strip, and wherein the blood sampling assembly and test assembly comprise separate units, the test strip is optionally stored in the test assembly and passed into the blood sampling assembling for contacting with the blood sample at the reaction location, wherein the test strip after contacting with the blood sample is moved to the test assembly for quantification of lactic acid concentration. In some such embodiments, the test assembly comprises a pay-out spool for passing the test strip to the blood sampling assembly and a reel-in spool for returning the test strip to the test assembly.

In some embodiments, the extent of a reaction between the reagent and a blood sample at the reaction location provides a quantitative indication of the concentration of lactic acid in a blood sample. Such a reaction can optionally be measured by measurement of changes in a parameter selected from the group consisting of optical parameters and electrical parameters of the blood sample and reagent mix. In embodiments where an optical parameter is measured, the test assembly further comprising a light source and a light detector, wherein the light detector detects changes in optical parameters of light from the light source transmitted through or reflected by the blood sample following reaction with the reagent.

In some embodiments, the device optionally further comprises one or more of a control system for controlling and adjusting the blood sampling assembly and the test assembly, a display for displaying measured lactic acid concentration, an alarm for signaling a lactic acid concentration outside a preset limit, and an alarm for signaling depletion of reagent.

In some embodiments, there is provided a method for monitoring fetal stress by automatically and repeatedly measuring a lactic acid concentration in a blood sample from a fetus, the method comprising providing a device as described above, attaching the blood sampling assembly to an organ of the fetus to extract a blood sample; conveying the blood sample to the reaction location; providing the reagent to the reaction location, wherein the reagent reacts with the blood sample; measuring the extent of reaction of the reagent with the blood sample; and calculating the concentration of lactic acid in the blood sample according to the extent of reaction of reagent with blood sample.

Additional features and advantages of the invention will become apparent from the following drawings and description.

### BRIEF DESCRIPTION OF THE DRAWINGS

For a better understanding of the invention with regard to the embodiments thereof, reference is made to the accompanying drawings, in which like numerals designate corresponding elements or sections throughout, and in which:

FIG. 1 shows a lactic acid monitoring device connected to a fetus;

FIG. 2 shows a probe of the lactic acid monitoring device, comprising a blood sampling assembly;

FIG. 3 shows a blood sampling assembly;

FIG. 4 shows a test assembly with a test strip;

FIG. 5A shows a miniaturized probe with a liquid reagent;

FIG. 5B shows a test assembly for miniaturized probe with a liquid reagent;

FIG. 6A shows a miniaturized probe with internal test strip;

FIG. 6B shows a test assembly for miniaturized probe with internal test strip;

FIG. 7A shows a blood sampling probe with external test strip; and

FIG. 7B shows a test assembly for a blood sampling probe with external test strip.

### DETAILED DESCRIPTION OF THE PREFERRED EMBODIMENTS

The present invention provides a device and method for monitoring of fetal stress by automatically repeated measurement of fetal blood lactic acid concentration.

The device of the present invention comprises a blood sampling assembly for obtaining a microvolume blood sample from a fetus, and a test assembly for quantifying lactic acid in a blood sample, the test assembly comprising a reaction location in fluid communication with the blood sampling assembly, and a reagent reservoir in communication with the reaction location, wherein the device is configured for automatically refreshing the reaction location with a blood sample through the blood sampling assembly, and for automatically refreshing the reaction location with reagent from the reagent reservoir.

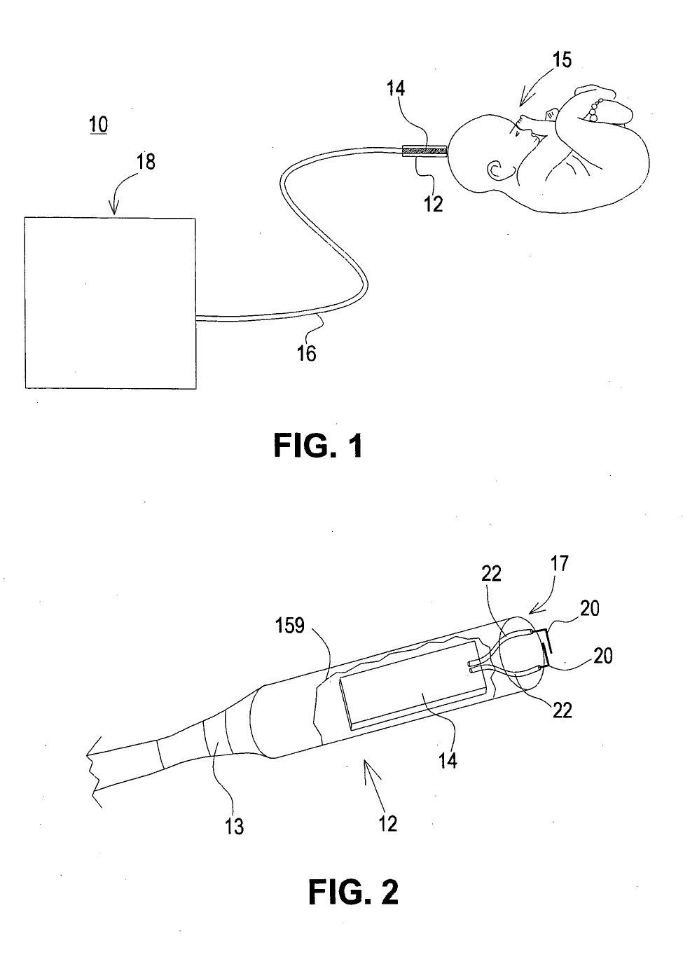

Referring now to FIG. 1, there is shown a representation of a fetal lactic acid monitor device 10, comprising a test assembly 18 and a blood sampling assembly 14, attached to the scalp of a fetus 15. Blood sampling assembly 14 is contained within a probe 12, configured for insertion into the vagina of a woman in labor, in order to contact the presenting portion of the body of fetus 15.

Preferably, probe 12 has an outside diameter of no more than 15-20 mm. Blood sampling assembly 14 is connected to test assembly 18 by a flexible protective conduit 16, in which are disposed the tubes and control wires required for operation of the system, as described below.

The test assembly 18 contains a reservoir configured for containing a reagent sensitive to lactic acid.

The reagent may be provided in the form of a liquid reagent, or incorporated into a test strip. In embodiments wherein a liquid reagent is used, the reaction location preferably comprises a reaction chamber within the test assembly 18, and the reagent is preferably contained in a reservoir in the test assembly 18 prior to use, wherein the reagent used for testing is automatically pumped to the reaction chamber from the reservoir after each measurement.

In embodiments wherein the reagent is incorporated within a test strip, the reaction location is the portion of the test strip which contacts the blood sample, and the reservoir comprises a spool of test strip paper, plastic or fabric comprising the reagent.

Throughout the monitoring process, the device 10 is maintained in a suitable condition for enabling receipt of a subsequent blood sample, either by providing a system for flushing residual blood after each measurement, or by providing the test assembly 18 at a very short distance from the point of blood extraction, such that no blood clotting takes place in the device.

The inventive device can repeatedly measure fetal blood lactic acid concentration, without piercing the fetus scalp every time a sample of blood is taken, thereby eliminating multiple wounds. The device 10 requires a single initial setup for each monitoring process, after which continuously repeated measurements can be obtained at predetermined, or adjustable time intervals, throughout the monitoring process, without the need to stop operation of the device for cleaning or removal of clotted blood, or refreshing of reagent.

According to some embodiments, the device is preset to withdraw a minimal amount of blood, which is preferably in the range of from about 3 μl to about 9 and more preferably from about 5 μA to about 7 pa, in each sampling, thereby minimizing the amount of blood withdrawn from the fetus.

According to some embodiments, the device 18 provides repeated measurement of fetal lactic acid concentration at preset time intervals. Preferably, measurements are repeated at time intervals in the range of from about 0.5 minutes to about 20 minutes, and more preferably about every 5 minutes. The measurement frequency can be decided upon according to the birth process and adjusted by the attending physician.

The blood sampling assembly 14 is configured for removable attachment to a part of the body of a fetus 15 from which a blood sample is to be withdrawn. Preferably, the blood sampling assembly 14 is configured for attachment to the part of the body of the fetus 15 presenting in the birth canal.

In some embodiments, wherein the fetus 15 is in cephalic presentation, the part of the body is the scalp. Alternatively, the part of the body may be the buttock or foot (for breech presentations).

In some embodiments, the blood sampling assembly 14 is contained within a probe 12 for contacting the blood sampling assembly 14 with the subject. The probe 12 may be an elongated probe, configured for insertion within the vagina of a woman in labor in order to bring the blood sampling assembly into contact with the presenting portion of the body of the fetus. Optionally, a flexible sleeve is provided over the proximal end of the probe 12 to permit flexible movement of the probe.

The distal end of the blood sampling assembly 14 is provided with at least one sharp, hollow pin having a diameter in the micrometer range. The pins are able to withdraw small amounts of blood by capillary action, and also serve as an anchoring mechanism for securing the blood sampling assembly 14 to the site of blood withdrawal from the body of the fetus 15. The inside diameter of the pins is preferably in the range of from about 100 to about 200 micrometers, so as to minimize the volume of blood withdrawn from the fetus.

The pins may be formed from, or coated with, any biocompatible material, such as stainless steel, silicon dioxide, titanium, gold, nickel, and the like. The pins may optionally be provided with a coating of an anti-coagulant, such as heparin, to prevent blood clotting.

The pins are generally curved, and may have, for example, a sickle or corkscrew shape to prevent accidental removal from the anchoring site. The device may comprise one or more pins. Preferably, two pins are provided.

According to some embodiments, the pins are retractable. During initial insertion of the probe into the vagina of the woman in labor, the retractable pins are in the retracted position. Once the distal end of the probe is positioned adjacent to the presenting part of the body of the fetus, the pins are extended outwards to penetrate a blood vessel of the fetus. Extension may be achieved by a screw mechanism, which is optionally manually controlled by the attending physician, or by any suitable extension mechanism.

Blood drawn from a tissue of the fetus 15 by the pins is transferred into the blood sampling assembly via inlet tubes by the operation of a pump. The pump may be any pump known in the art which is suitable for pumping small volumes of blood, and may be, for example, electrically, pneumatically or hydraulically operated. Optionally, the pump may be provided with a unidirectional valve to prevent liquid used in testing from leaking back into the body of the fetus 15.

The test assembly 18 comprises a system for determining lactic acid concentration in a blood sample. At a reaction location, components of lactic acid in the blood react with one or more components of a reagent, providing a chemical reaction the extent of which is indicative of the quantity of lactic acid in the blood sample. Typically, such reactions are chromatogenic reactions, for example where the intensity of a generated color is related to the concentration of lactic acid in the blood sample.

Any lactic acid quantification system known in the art may be used for this purpose. For example, optical measurement systems may be used, based on use of a reagent which changes its optical properties according to the lactic acid concentration in the blood sample, such as, for example, nicotinamide adenine dinucleotide (NAD) lactate dehydrogenase.

According to some embodiments, a test strip may comprise any lactic acid test paper, plastic or fabric known in the art, which can be provided in the form of a reel for disposing on a spool. Examples include the Reflectoquant® Lactic Acid, (Catalog No. 16127/1, by Gallade Chemical Inc., USA, California) which is based on the fact that lactic acid (lactate) is oxidized by nicotinamide adenine dinucleotide (NAD) under the catalytic effect of lactate dehydrogenase to form pyruvate. In the presence of diaphorase, the NADH formed in the process reduces a tetrazolium salt to form a blue formazan, the concentration of which is determined by its optical properties, by reflective or transmissive color measurement. In some embodiments, the blood sampling assembly 14 comprises a modification of an automated blood sampling system, such as the AccuSampler® (DiLab, Sweden).

In some embodiments, the test assembly 18 comprises a reagent reservoir in the form of a roll of test strip disposed on at least one spool, in order to provide continuous feed of unused test strip, wherein the reagent for quantification of lactic acid is incorporated into the test strip. In such embodiments, the test assembly 18 optionally further comprises a second spool onto which portions of test strip are wound after use. Optionally, there is provided an audible and/or visual alarm which is activated when the supply of unused test strip is almost depleted.

In some embodiments, the test assembly 18 comprises a reservoir for containing a liquid reagent prior to use, a measurement chamber in which liquid reagent is reacted with the blood sample, and a tube and pump for transferring the liquid reagent from the reservoir to the measurement chamber.

In some such embodiments, cleaning fluid from a cleaning fluid reservoir is preferably pumped into the measurement chamber and the blood input tube after reaction has taken place, for cleaning of the chamber and tubes. The cleaning fluid preferably comprises saline.

According to some embodiments, the test assembly 18 further comprises a light source and a light detector for measurement of changes in light reflection or transmission following reaction of the blood sample with the reagent. The test assembly 18 preferably further comprises a processor for calculating the lactic acid concentration in the blood sample based on the optical properties of the sample, and a monitor for displaying the lactic acid concentration readings. Optionally, the test assembly 18 further comprises an audible and/or visual alarm which is activated if the lactic acid concentration is outside a preset limit.

It will be appreciated by those skilled in the art that as an alternative to optical systems, other systems for quantification of lactic acid may be used, such as those based on changes in electrical parameters such as capacitance, resistance, and conductance.

For embodiments using liquid reagent, spectrophotometer devices, such as “BioPhotometer plus” (Eppendorf, Hamburg, Germany) may be used.

In some embodiments, the blood sampling assembly 14 and the test assembly 18, comprising either test strip or liquid reagent, are provided within a single housing, such as, for example a miniaturized probe. In such a miniaturized probe, pump dimensions are preferably in the range of from about 3 mm to about 5 mm.

Alternatively, the blood sampling assembly 14 and the test assembly 18 are provided as separate units.

In some embodiments wherein the blood sampling assembly 14 and the test assembly 18 are provided as separate units, the blood sampling assembly and the test assembly are in fluid communication with each other, by use of a conduit such as a flexible blood input tube, such that blood withdrawn from the subject by the blood sampling assembly 14 may be transferred to the test assembly 18 for quantification.

According to some embodiments wherein the blood sampling assembly 14 and the test assembly 18 are provided as separate units, blood samples may be transferred from the blood sampling assembly 14 to the blood test assembly 18, by use of a column of saline. In such embodiments, in addition to a blood pump for transferring blood from the hollow pins to the blood sampling assembly, the blood sampling assembly further comprises a saline pump.

According to some embodiments, saline is pumped into the blood sampling assembly 14 via a saline input tube, from a reservoir which is optionally contained within the test assembly. The saline input tube and the blood inlet tubes, together with a blood output tube, meet at a junction within the blood sampling assembly 14. The saline input tube and the blood output tube each have diameters in the range of from about 1 mm to about 1.5 mm. Blood from the inlet tubes first enters the blood output tube, followed by saline from the saline input tube, such that a column of pumped saline pushes the blood sample along the blood output tube to the test assembly 18. This enables transfer of microvolume quantities of blood, with mixing of saline and blood during the process being prevented by the use of tubes of suitably small diameter.

The saline also serves to flush residual blood from the blood input tube, such that the device is in a suitable condition to receive a subsequent blood sample without the need to stop operation of the device to clean the blood input tube.

According to some embodiments, the test assembly 18 further comprises a suction pump for removing liquid from the blood input tube via a clearing tube, and an optical detector which distinguishes between saline and blood, such that when the optical detector identifies the presence of a sample of blood, a signal is transmitted to the suction pump to cease operation once the head of the sample of blood is calculated to have reached the opening of the clearing tube, such that blood is allowed to flow to an open end of the blood input tube.

A test strip is provided adjacent to the open end of the blood input tube, such that blood flowing from the open end of the blood input tube contacts the test strip at a reaction location.

As an alternative to saline, any suitable inert and biocompatible liquid may be used. However, saline is generally preferred, as is it commonly available in the medical environment and its use does not require any special approval from a governmental authority.

In some embodiments wherein the blood sampling assembly 14 and the test assembly 18 are provided as separate units, a test strip is stored in the test assembly 18, and passes into the blood sampling assembly 14 for contacting with the blood sample at the reaction site. The test strip containing the reaction site is then returned to the test assembly for quantification of blood lactate acid quantification. The test assembly 18 may further comprise a first pay-out spool on which the test tube is disposed, for feeding the test strip to the blood sampling assembly via a test strip input tube, and a second reel-in spool for drawing the test strip back to the test assembly 18 via a test strip output tube.

The tubes and wires required for operation of the system are preferably contained within a flexible conduit connecting the blood sampling assembly 14 and the test assembly 18, and covered by the flexible sleeve disposed upon the proximal end of the probe.

According to some embodiments, wherein the blood sampling assembly 14 and the test assembly 18 are provided within separate housings, the reaction location is on the test strip in the blood sampling assembly 14, and the test strip then advanced such that the portion of test paper containing the reaction location is transferred to the test assembly 18 for measurement.

According to some embodiments, wherein the blood sampling assembly 14 and the test assembly 18 are provided within a single housing, the reaction location is located close to the site of blood extraction, such that no clotting of blood occurs in the device.

Operation, such as activation/deactivation and timing of components, such as pumps and valves, is controlled by standard hardware and software systems, as known in the art.

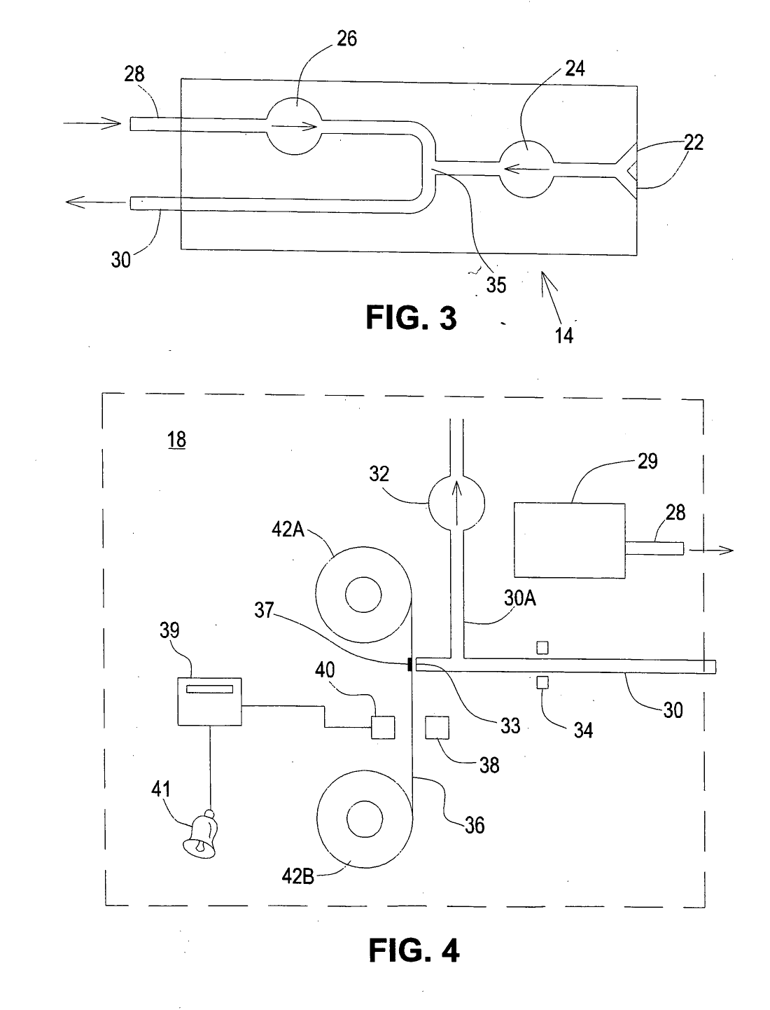

FIG. 2 shows blood sampling assembly 14 disposed within probe 12, as a cutaway view of cutout portion 159. The distal end 17 of blood sampling assembly 14, is provided with hollow pins 20 for attachment to the fetus. Blood is drawn through hollow pins 20 by capillary action, and passes to blood sampling assembly 14 via blood inlet tubes 22, by operation of a blood pump 24 (shown in FIG. 3). Flexible sleeve 13 is disposed upon the proximal end of probe 12 in order to allow flexibility of the probe movement.

FIG. 3 shows representation of blood sampling assembly 14, comprising a blood pump 24 which pumps blood from pins 20 into blood sampling assembly 14 via tubes 22, and a saline pump 26, which pumps saline from a saline reservoir 29 located in test assembly 18 (shown in FIG. 4), via saline input tube 28. Alternatively, saline pump 26 may be located inside test assembly 18.

Saline input tube 28 is connected at junction 35 to blood inlet tubes 22 via blood pump 24. Blood arriving at junction 35 via tube 22 through blood pump 24 is pushed by a column of saline into blood output tube 30. Blood output tube 30 leads blood from blood sampling assembly 14 to test assembly 18. Tubes 28 and 30 are inserted within sleeve 13 and conduit 16.

Pumps 24, 26 have outside diameters of 3-5 mm and tubes 28, 30 have outside diameters of 1-1.5 mm.

Alternatively, Micro Electro Mechanical Systems (“MEMS”) technology may be utilized to construct the blood sampling assembly 14 as a monolithic unit.

Pumps 24, 26 may be electrically, pneumatically or hydraulically controlled and operated. These types of control are well understood by those skilled in the art and are all not shown in the drawings.

FIG. 4 shows a schematic representation of test assembly 18. Blood is transferred from blood sampling assembly 14 to test assembly 18 via blood input tube 30, having an open distal end 33. Test assembly 18 comprises a suction pump 32 which removes liquid from blood output tube 30 via a clearing tube 30A to a waste reservoir (not shown), and an optical detector 34, which distinguishes between blood and saline in blood output tube 30. Upon detection by optical detector 34 of the presence of a blood column at a specific point along the length of blood output tube 30, a signal is transmitted to suction pump 32 such that suction pump 32 ceases operation once the head of the blood column is calculated to have reached the point at which blood output tube 30 connects with clearing tube 30A, such that all saline (and a minimal amount of blood from the head of the blood sample) is removed into clearing tube 30A, while blood alone is allowed to continue flowing to the open end 33 of blood output tube 30.

Test assembly 18 is provided with lactic acid test strip 36 (such as that described by Bell et al in U.S. D512,512) located adjacent to open end 33 of blood output tube 30, such that blood flowing to open end 33 wets test strip 36.

Test strip 36 is supported by two spools 42A, 42B, located on opposite sides of open end 33 of blood output tube 30. Spool 42A is a pay-out spool on which test strip 36 is stored prior to use, and which advances unused portions of test strip 36 to open end 33 of blood output tube 30. Spool 42B is a reel-in spool, onto which test strip 36 is wound after use.

Spool 42A preferably carries sufficient test strip 36 for a few hours of operations, equal to several tens of measurements. Preferably, spool 42A is provided with an audible and/or visual alarm which is activated when the end of test strip in spool 42A is about to be reached, to signal the need for a replacement.

When the end of the blood sample in blood output tube 30 is detected by optical detector 34, a further signal is transmitted to activate suction pump 32, such that the saline column in blood output tube 30 is removed to prevent saline from diluting the blood on test strip 36.

Upon reaching open end 33 of blood output tube 30, the blood sample obtained from the fetus contacts test strip 36 at reaction location 37. Following reaction of the blood with test strip 36 at reaction location 37, the concentration of lactic acid at reaction location 37 is determined optically.

Test assembly 18 further comprises a light source 38 and a light detector 40. An actuator (not shown) moves the used test strip towards spool 42B so the reaction location 37 is situated between light source 38 and light detector 40. Light source 38 is directed towards reaction location 37 on the face of test strip 36 which contacted the blood sample. Light detector 40 is positioned on the opposite side of test strip 36 from light source 38, and provides a measurement of the optical properties of reaction product formed upon reaction of the reagent in test strip 36 with the blood sample, as a function of light transmission, which is calibrated to lactic acid concentration in the blood sample. Alternatively, light reflection can be used to provide the measurement of the reacted reagent if the reflective properties of the reagent are changed upon reaction with the blood sample.

A standard calibration paper (not shown) can be used in place of test strip 36 to periodically calibrate the optical measurement.

Light detector 40 is connected by wired or wireless communication to a monitor 39 for display of lactic acid concentration in the blood.

Monitor 39 is optionally provided with an audible and/or visual alarm 41, which is activated upon lactic acid concentration in the blood sample rising above a preset threshold value.

Referring now to FIGS. 1-4, the measurement process is conducted as follows. After hollow pins 20 of probe 12 are attached to fetus 15, the device is activated to begin the test cycle. The cycle begins with activation of saline pump 26 and suction pump 32 to flush any previous contents of blood output tube 30 via clearing tube 30A. Saline pump 26 may be programmed to stop after a preset time, depending on the length of blood output tube 30, or alternatively, upon a signal from blood detector 34 that no residual blood is detected in blood output tube 30.

Blood pump 24 is then activated to draw new blood from hollow pins 20 via inlet tubes 22 into blood output tube 30. The volume of blood can be preset, and is usually such that the dead volume of blood from pins 20, inlet tubes 22, and pump 24, plus the volume of blood required for sampling, i.e. from about 5 μl to about 7 μl, is pumped beyond the junction point 35, and then stops operation. Saline pump 26 is then reactivated, drawing saline from saline reservoir 29 via saline input tube 28, to junction 35, from which point the saline pushes the blood sample via blood output tube 30 to test assembly 18, a distance of about 50-100 cm.

Suction pump 32 clears saline from blood output tube 30 before the saline reaches open end 33, such that it does not wet reaction strip 36. After a predetermined time delay from the time at which optical detector 34 detects the presence of blood in blood output tube 30, operation of suction pump 32 is stopped, such that blood continues to open end 33 of blood output 30 and wets test strip 36. The time delay is calculated to remove from blood output tube 30 all saline, plus the dead volume of blood from pins 20, inlet tubes 22, and pump 24, which constitute blood from the previous measurement, thereby allowing only fresh blood to reach open end 33 of blood input tube 30. Suction pump 32 is reactivated once the end of the blood sample arrives at open end 33 of blood output tube 30, to prevent dilution of the blood sample with saline. Saline pump 26 is reactivated for a time period which may be preselected, to flush blood residues from blood output tube 30 prior to the next measurement cycle.

The blood sample contacts test strip 36 at reaction location 37, wherein reaction with the reagent in test strip 36 causes a change in the optical properties of test strip 36 at reaction location 37, as measured by changes in reflection or transmission of light emitted by light source 38, and detected by light detector 40. In the case of reflection measurement, light source 38 and light detector 40 may be located on the same side of test strip 36 (not shown). The extent of the change in reflection or transmission is related to the concentration of lactic acid in the blood. Readings obtained by light detector 40 are processed to obtain a reading of lactic acid concentration, which is displayed on monitor 39. Alarm 41 is activated if the lactic acid concentration is outside a preset limit.

The test cycle is repeated at pre-determined time intervals, as determined by the attending physician, such as, for example, every 5 minutes.

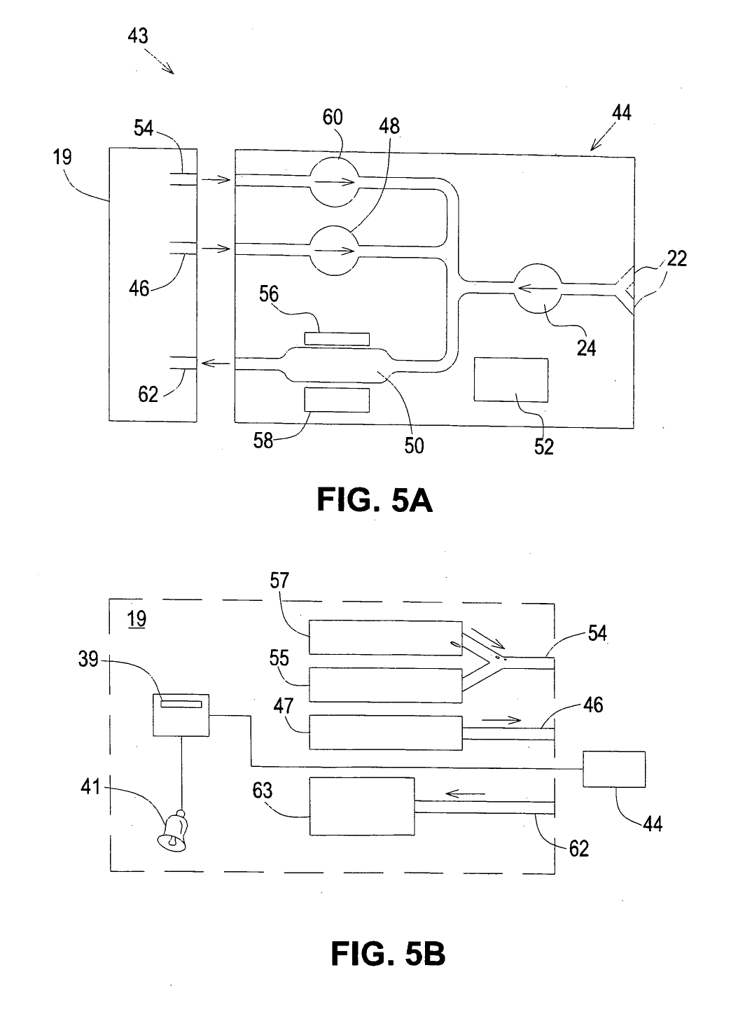

FIG. 5A shows a representation of device 43, an alternative embodiment of the present invention wherein the blood sampling assembly and the test assembly are contained within measurement probe 44. Measurement probe 44 is preferably constructed using MEMS technology, which can integrate flow channels for liquids, pumps, electronics and optical components in small and mass producible units. In this alternative embodiment, a liquid reagent which produces a color change upon contact with lactic acid in blood, is used instead of a test strip.

Measurement probe 44 comprises inlet tubes 22, blood pump 24, reagent or calibration fluid pump 60, measurement chamber 50, ultrasound transducer 52, saline pump 48, light source 56, and optical detector 58.

Device 43 further comprises a support unit 19. Ports 54, 46, and 62 provide fluid communication between measurement probe 44 and support unit 19, preferably via flexible tubes disposed inside a flexible sleeve (not shown) together with electrical wiring.

FIG. 5B shows a representation of support unit 19, comprising waste receptacle 63, reagent reservoir 57, saline reservoir 47, and calibration fluid reservoir 55. Support unit 19 further comprises monitor 39 for display of calculated lactic acid concentration, and alarm 41 which is activated if the lactic acid concentration is outside a preset limit.

Measurement probe 44 is attached to the fetus using hollow pins as described above for previous embodiments. Upon activation of measurement probe 44, saline pump 48 draws saline from saline reservoir 47 in support unit 19, via port 46 to measurement chamber 50. The saline flushes any previously used reaction mixture from measurement chamber 50, via exhaust port 62, to waste receptacle 63, thus cleaning measurement chamber 50 in preparation for the new measurement. It should be noted that any suitable cleaning fluid may be used instead of saline in this context. After a suitable time period, saline pump 48 ceases operation. Reagent pump 60 then pumps reagent liquid via inlet tube 54 from reservoir 57 into measurement chamber 50.

Blood pump 24 commences pumping a new blood sample from fetus 15 via inlet tubes 22. After about 5-7 μl of blood is withdrawn from the fetus, and pumped to measurement chamber 50, blood pump 24 is stopped. An ultrasound transducer 52 or any other miniature mixing device is used for thorough mixing of blood and reagents. Serpentine or binary shuffling tubes (not shown) may be used to mix the blood sample.

After a suitable period of time is allowed for the reaction to take place, the optical measurement is performed, using light source 56 and detector 58, placed on opposite sides of the measurement chamber 50, and the lactic acid concentration in the blood sample is transmitted to monitor 39, by wired or wireless communication, as described above for previous embodiments. Transmissive or reflective optical measurements, or electrical measurements are suitable for this purpose. Known spectrophotometer technology, such as “BioPhotometer plus” (Eppendorf, Hamburg, Germany) may be used.

After the measurement ends, saline pump 60 is used to draw saline from saline reservoir 57, situated in support unit 19, via port 54, to flush the reaction mixture from measurement chamber 50 via exhaust port 62 leading to waste receptacle 63. This cleans measurement chamber 50 and prepares it for the next measurement, and ends the measurement cycle.

According to some embodiments, calibration fluid reservoir 55 is periodically used to supply calibration fluid to measurement chamber 50 to calibrate the light source 56 and detector 58. In such embodiments, pump 60 is used for either pumping reagent or calibration fluid, with the selection controlled by use of valves (not shown) provided near the connection point between the tubes leading out of reservoirs 55 and 57.

Optionally, a unidirectional valve may be incorporated in line with blood pump 24 or as part of blood pump 24 to prevent leakage of reagent or saline liquids into the body of the test subject. More than one unidirectional valve may be incorporated, using different technologies, for safety reasons, to minimize the probability of reagent or calibration fluids reaching the fetus.

According to some embodiments, a controlled, unidirectional valve (not shown) may be used instead of pumps 24, 26, and 60, and suction means at port 62, and valves at ports 54 and 46 may be used to conduct the measurement process described above.

In some embodiments, several different reagent liquids may be used to sequentially measure different properties of the blood (or any other bodily liquid), for example glucose level, pH, etc.

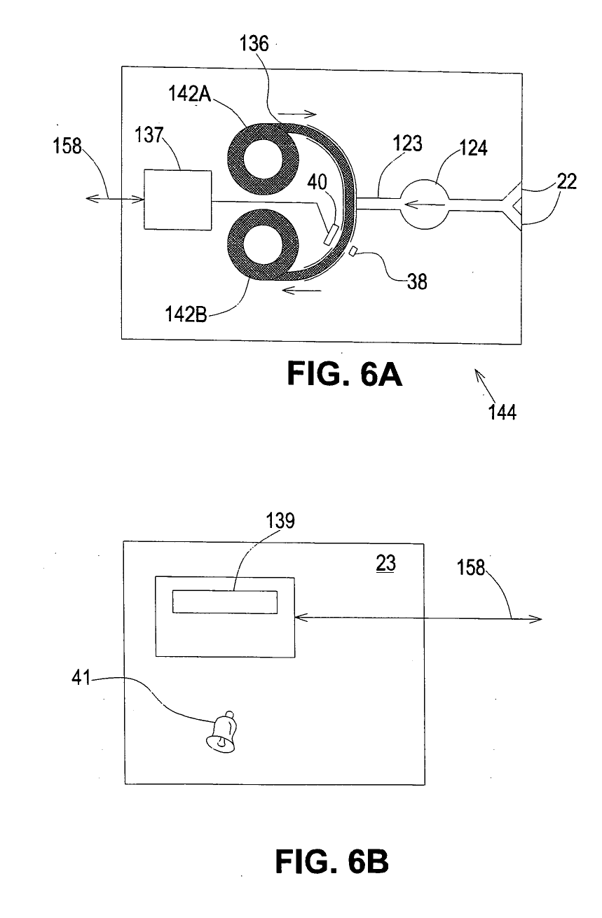

FIG. 6A shows a representation of an alternative embodiment of the present invention, wherein the test assembly and the blood sampling assembly are provided within a probe 144, comprising an internal test strip 136. Probe 144 is preferably a miniaturized probe constructed using MEMS technology.

Probe 144 comprises inlet tubes 22, for conveying blood from hollow pins 20 (as described with regard to previous embodiments), blood pump 124, blood outlet tube 123, light source 38, light detector 40, internal control unit 137, test strip 136, test strip pay-out spool 142A, and test strip reel-in spool 142B.

Probe 144 is connected by wireless or wired communication 158 to support unit 23, shown in FIG. 6B, which comprises a screen 139, an alarm 41, and control means (not shown), for medical personnel to control the operational parameters of the device.

Probe 144 may be disposable or may be sterilized after each use. When sterilized, only test strip 136 needs to be replaced. Test strip 136 is long enough to provide sufficient test paper for readings to be taken over a period of 24-48 hours, in the event of a prolonged birth process.

In this embodiment, the measurement process is conducted as follows. Probe 144 is attached to fetus 15, as previously described, and the device is activated. Blood pump 124 pumps a 5-7 μl blood sample from fetus 15 via inlet tubes 22. The blood sample may be detained in a chamber (not shown) in blood pump 124, and then released through a valve (not shown) via blood outlet tube 123, directly onto test strip 136. Test strip 136 may be made from paper, plastic or fabric, and includes a reagent that changes its optical properties according to the lactic acid concentration in the blood sample. Test strip 136 is spooled from pay-out spool 142A to reel-in spool 142B.

An actuator (not shown) moves the portion of test strip 136 containing the blood sample towards spool 142B so the blood sample is situated between light source 38 and light detector 40. The optical properties are measured. Note that a transmissive optical measurement is shown, but reflective optical measurement can be used as well, depending on the design of the test strip and the reagent in it.

A standard calibration strip (not shown) can be used in place of test strip 136 to periodically calibrate the optical measurement. Alternatively, reagent and calibration materials can be located on separate portions along the length of test strip 136.

Light source 38 and light detector 40 are used, with internal control unit 137, to measure the blood lactic acid concentration. The results are processed and transmitted via wireless or wired communication 158 to appear on screen 139: Alarm 41 is used to indicate readings out of the normal range. This ends the test cycle and the process is repeated at pre-determined time intervals, for example every 5 minutes.

An advantage of this particular embodiment is that there is no need for repeated washing of the apparatus, as the presenting portion of the fetus is very close to the test strip, such that blood does not need to travel far until it reaches the test strip, thus, blood coagulation problems are avoided. If a small amount of blood is coagulated at the open end of blood outlet tube 123, the action of pump 124 will push the coagulated blood towards test strip 136 at the beginning of the process.

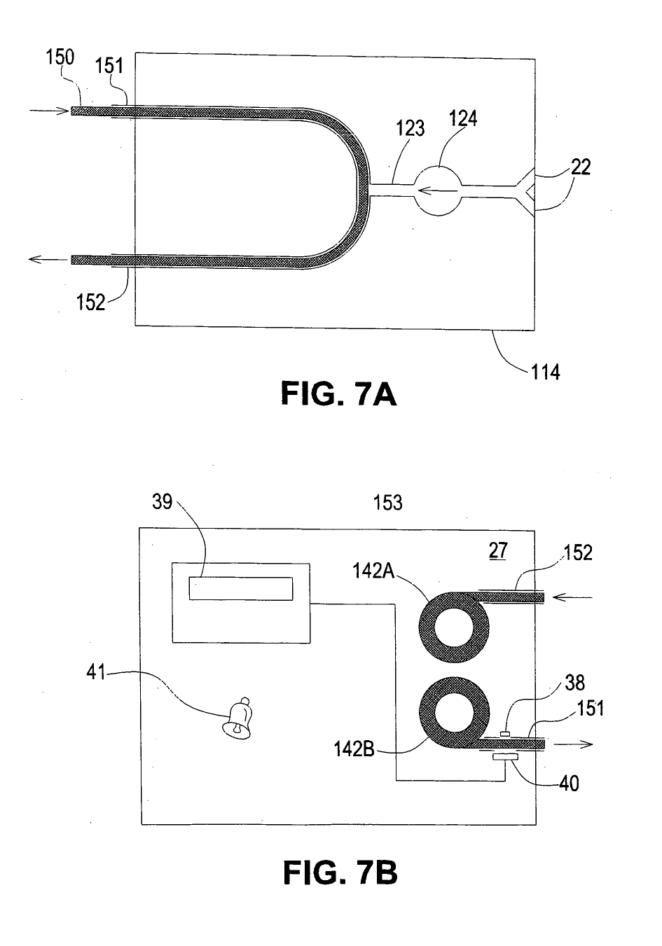

FIGS. 7A and 7B show representations of an alternative embodiment of the present invention, comprising a blood sampling probe 114 and a test assembly 27.

As shown in FIG. 7A, blood sampling probe 114 comprises inlet tubes 22, a blood pump 124, blood outlet tube 123, test strip 150, test strip inlet tube 151, and test strip outlet tube 152.

FIG. 7b shows test assembly 27, comprising test strip 150, test strip inlet tube 151, test strip outlet tube 152, test strip pay-out spool 142A, test strip reel-in spool 142B, light source 38, light detector 40, monitor 39 and alarm 41.

In this embodiment, the measurement process is conducted as follows. Blood sampling probe 114 is attached to fetus 15, as described previously, and the device is activated. Blood pump 124 pumps a 5-7 μl blood sample from the fetus via inlet tubes 22. The blood sample may optionally be detained in a chamber (not shown) in blood pump 124, and then released through a valve (not shown) into blood outlet tube 123 directly onto test strip 150. Test strip 150 may be made from paper, plastic, or fabric, and includes a reagent that changes its optical properties according to the lactic acid concentration in the blood sample. Test strip 150 is spooled from spool 142A to spool 142B and is advanced as described below.

An actuator (not shown) moves the portion of test strip 150 containing the blood sample through test strip outlet tube 152 to test assembly 27 so the portion of test strip 136 containing the blood sample is situated between light source 38 and light detector 40. Light is transmitted via an opening 153 in test strip inlet tube 151, and the optical properties are measured. Note that a transmissive color measurement is shown, but reflective color measurement can be used as well, depending on the design of the test strip and the reagent in it.

The test strip 150 may be very long but only a part of it will be used. Optionally, reagent is not present along the entire length of test strip 150 but only on selected areas. The areas with the reagent are separated from one another by a distance that is greater than the distance between blood sampling probe 114 and test assembly 27. The area with the reagent may be marked electrically (such as by a conductive portion) or optically (such as by reflective silvering, white or black marking), for sensing by optical sensors present at two locations: the area where the blood wets the strip and the area where the optical measurement is performed.

The reading of the lactic acid concentration is displayed on monitor 39, and can be sent electronically to processing units, patient monitoring equipment etc. via electronic or optical interconnects.

An alarm 41 is activated if the lactic acid concentration exceeds a preset upper limit.

This ends the test cycle and the process is repeated at time intervals determined by the attending physician, for example every 5 minutes.

All embodiments in this invention are described as used for measurement of lactic acid concentration in fetal blood, but the same may be used for repeated sampling and measurements of any blood component in any human patient or animal. In addition, urine or other bodily liquid can be sampled and measured.

Although particular embodiments of the invention have been described and illustrated herein, it is recognized that modifications and variations may readily occur to those skilled in the art and consequently, it is intended that the claims be interpreted to cover such modifications and equivalents.

1. A device for monitoring fetal stress by automatically and repeatedly measuring a lactic acid concentration in a blood sample from a fetus, the device comprising:
a blood sampling assembly for attachment to a fetal organ to extract a microvolume blood sample; and
a test assembly for quantifying said lactic acid concentration in said blood sample extracted by said blood sampling assembly,
said test assembly comprising a reaction location in fluid communication with said blood sampling assembly and a reagent reservoir in communication with said reaction location, said reagent reservoir comprising a reagent sensitive to said lactic acid concentration,
wherein interaction between said blood sample and said reagent provides a measureable indication of blood lactic acid concentration,
wherein the device is configured for automatically refreshing said reaction location with a blood sample through said blood sampling assembly and for automatically refreshing said reaction location with reagent from said reagent reservoir.

2. The device of claim 1, wherein said blood sampling assembly and said test assembly are provided as separate units.

3. The device of a claim 1, wherein said blood sampling assembly is contained within a probe for providing contact between said blood sampling assembly and the fetus.

4. The device of claim 3, wherein said blood sampling assembly and said test assembly are provided within said probe.

5. The device of claim 4, wherein said reaction location is provided proximal to said blood sampling assembly.

6. The device of claim 1, wherein said reagent reservoir comprises a reservoir for containing a liquid reagent.

7. The device of claim 1, wherein said reagent reservoir comprises a reel of test strip wherein said reagent is incorporated into said test strip.

8. The device of claim 7, wherein said reel of test strip is disposed on at least one spool for providing continuous feed of unused test strip.

9. The device of claim 8, wherein said test assembly further comprises a second spool wherein portions of said test strip are wound onto said second spool after use.

10. The device of claim 6, wherein said reaction location comprises a reaction chamber provided within said test assembly.

11. The device of claim 7, wherein said reaction location is a portion of said test strip which contacts a blood sample from said blood sampling assembly.

12. The device of claim 1, wherein a distal end of said blood sampling assembly comprises at least one sharp hollow pin for reversible attachment to the body of a fetus.

13. The device of claim 12, wherein said at least one pin has an internal diameter in the range of from about 100 to about 200 micrometers.

14. The device of claim 12, wherein said at least one pin is coated with an anti-coagulant to prevent blood clotting.

15. The device of claim 12, wherein said device can be preset to withdraw a blood sample of specified volume from the fetus via said at least one hollow pin.

16. The device of claim 15, wherein said specified volume in the range of from about 5 μl to about 7.

17. The device of claim 1, wherein said device is preset to repeatedly withdraw a blood sample from said fetus at a specified time interval.

18. The device of claim 17, wherein said specified time interval is in the range of from about 0.5 minutes to about 20 minutes.

19. The device of claim 18, wherein said time interval is about 5 minutes.

20. The device of claim 12, wherein said blood sampling assembly comprises at least one blood pump for pumping blood from said at least one pin to said blood sampling assembly.

21. The device of claim 6, wherein said test assembly further comprises a reagent pump for pumping said liquid reagent from said reagent reservoir to said reaction location.

22. The device of claim 2, further comprising a blood input tube for providing fluid communication between said blood sampling assembly and said test assembly.

23. The device of claim 22, further comprising a saline reservoir, a saline input tube in fluid communication with said blood input tube, and a saline pump for pumping saline from said saline reservoir to said blood input tube.

24. The device of claim 20, wherein any of said blood pump, said reagent pump and said saline pump have diameters in the range of from about 3 to about 5 mm.

25. The device of claim 22, wherein any of said blood input tube and said saline input tube has a diameter in the range of from about 1 mm to about 1.5 mm.

26. The device of claim 23, wherein said blood sample entering said blood input tube is pushed along said blood input tube to said test assembly by said saline pumped from said saline reservoir into said blood input tube.

27. The device of claim 23, wherein said saline pumped from said saline reservoir into said blood input tube flushes blood residues from said blood input tube after passage of said blood sample.

28. The device of claim 23, further comprising a cleaning fluid reservoir in fluid communication with said blood input tube, wherein said cleaning fluid from said cleaning fluid reservoir flushes blood residues from said blood input tube after passage of said blood sample.

29. The device of claim 26, said blood input tube comprising an open end adjacent to said test strip, wherein said test unit further comprises a suction pump for removing liquid from said blood input tube via a clearing tube having an opening at a branch point with said blood input tube, and an optical detector which distinguishes between said saline and said blood sample, such that when said optical detector identifies the presence of said blood sample, a signal is transmitted to said suction pump to cease operation once said blood sample is calculated to have reached said opening at said branch point with said blood input tube, such that said blood sample is allowed to flow to said open end of said blood input tube and contact said test strip at said reaction location.

30. The device of claim 7, wherein said blood sampling assembly and said test assembly comprise separate units, wherein said test strip is stored in said test assembly and passed into said blood sampling assembling for contacting with said blood sample at said reaction location, wherein said test strip after contacting with said blood sample is returned to said test assembly for quantification of said lactic acid concentration.

31. The device of claim 30, wherein said test assembly comprises a pay-out spool for passing said test strip to said blood sampling assembly and a reel-in spool for returning said test strip to said test assembly.

32. The device of claim 1, wherein the extent of a reaction between said reagent and a blood sample at said reaction location provides a quantitative indication of the concentration of lactic acid in a blood sample.

33. The device of claim 32, wherein the extent of said reaction can be measured by measurement of changes in a parameter selected from the group consisting of optical parameters and electrical parameters.

34. The device of claim 33, wherein said parameter is an optical parameter, said test assembly further comprising a light source and a light detector, wherein said light detector detects changes in said optical parameters of light from said light source, transmitted or reflected from said blood sample following reaction with said reagent.

35. The device of claim 1, further comprising at least one of a control system for controlling and adjusting said blood sampling assembly and said test assembly, a display for displaying said measured lactic acid concentration, an alarm for signaling a lactic acid concentration outside a preset limit, and an alarm for signaling depletion of said reagent.

36. A method for monitoring fetal stress by automatically and repeatedly measuring a lactic acid concentration in a blood sample from a fetus, the method comprising:
providing a device in accordance with any of the preceding claims;
attaching said blood sampling assembly to an organ of the fetus to extract a blood sample;
conveying said blood sample to said reaction location;
providing said reagent to said reaction location, wherein said reagent reacts with said blood sample;
measuring the extent of said reaction of said reagent with said blood sample; and
calculating a concentration of said lactic acid in said blood sample according to the extent of said reaction of said reagent with said blood sample.
